(19) United States
(12) Patent Application Publication
(10) Pub. No.: US 2020/0179556 A1
(43) Pub. Date: Jun. 11, 2020

(54) Ozone Generator for Flosser

(71) Applicant: Seemie N. Syed, Dania Beach, FL (US)

(72) Inventor: Seemie N. Syed, Dania Beach, FL (US)

(21) Appl. No.: 16/216,383

(22) Filed: Dec. 11, 2018

(51) Int. Cl. A61L 9/22; A61L 2/20; A61L 2/24; A61L 2/26

(52) U.S. Cl. CPC A61L 9/22; A61L 2/202; A61L 2/24; A61L 2202/15; A61L 2202/24; A61L 2202/11; A61L 2202/122; A61L 2/26

(57) ABSTRACT

An apparatus for performing dental hygiene includes a mechanism to sterilize a head portion based on an ozone generator integrated with a base of the apparatus. The ozone generator includes a corona discharge tube, a micro air pump to circulate air through the corona discharge tube, and a bubble diffuser to diffuse ozone in a fluid. The ozone diffused fluid is transferred to a space around the head portion to sterilize the head portion. The space around the head portion is created by a cap like device connected to the head portion in watertight manner. An ultrasonic mechanism is coupled to the cap like device as an additional sterilizing mechanism, and generates ultrasonic waves through the fluid held around the head portion for sterilizing the head portion.

### BACKGROUND

### Technical Field

The embodiments herein generally relate to dental devices. More particularly, they pertain to a dental device incorporating an ozone generator to add ozone to a fluid used in a bacteria-removing mechanism of the device.

### Description of the Related Art

Tooth decay has been implicated in the cause and progression of many systemic and organ-related diseases. Dental professionals recommend removal of dental plaque from the surface of teeth on at least a daily basis. Plaque is a clear sticky substance, which continually forms in the mouth above and below the “gumlines”. Plaque can calcify and form a hardened material, tartar; however, cleaning around each tooth prevents calcification of plaque. This cleaning aids in the prevention of cavities and related dental problems. It is well recognized that brushing with a toothbrush is not sufficient to avoid these problems.

Various types of flossing devices are available to remove plaque and clean around teeth to overcome the shortcomings of brushing with a toothbrush alone. However, during the process of flossing by a user, bacteria from the mouth of the user, or from other sources, may get deposited on head of the flossing device, and grow. Thus, a flosser used repeatedly may simply spread bacteria. What is needed is an apparatus that effectively sterilizes the head of the flossing device by incorporating additional agents for sterilization of the head of the flossing device.

### SUMMARY

In view of the foregoing, an embodiment herein provides apparatus for performing dental hygiene, comprising a head portion configured for performing one or more dental hygiene operations; and a sterilizing mechanism operatively connected to the head portion that reduces bacteria from the head portion. The sterilizing mechanism comprises an ultrasonic mechanism for reducing bacteria in the head portion of the apparatus.

The apparatus further comprises a base comprising; a reservoir to store a fluid used during the one or more dental hygiene operations performed using the head portion of the apparatus, as well as by the sterilizing mechanism to reduce bacteria from the head portion; a corona discharge tube to generate ozone; and a bubble diffuser operatively coupled to the reservoir, which receives ozone from the corona discharge tube and diffuses the received ozone with the fluid, when the fluid is being supplied from the reservoir to the sterilizing mechanism.

The corona discharge tube may comprise a cylindrical shape outer electrode; an inner electrode concentrically positioned within the outer electrode; a tube made of a dielectric material, such as but not limited to glass, positioned between the outer electrode and the inner electrode such that an air chamber is formed between the glass tube and the outer electrode; and a micro air pump to circulate air through the air chamber between the glass tube and the outer electrode.

The apparatus may comprise a power source to apply electric potential difference across the outer electrode and the inner electrode, and to supply electric power to the micro air pump.

The apparatus may comprise a cap like device operatively connected to the head portion, and the sterilizing mechanism may be configured with the cap like device. The cap like device may be adapted to fit the head portion in watertight manner to create a space around the head portion to hold the fluid from the reservoir around the head portion.

The ultrasonic mechanism of the sterilizing mechanism may generate ultrasonic waves through the fluid held around the head portion for sterilizing the head portion.

Another embodiment provides an ozone generator for configuring with an apparatus for performing dental hygiene, comprising a corona discharge tube operatively coupled to a base of the apparatus for generating ozone; and a bubble diffuser operatively coupled to a reservoir of the apparatus that holds a fluid used in the apparatus during dental hygiene operation. The bubble diffuser is operatively coupled to an outlet of the corona discharge tube to receive the generated ozone from the corona discharge tube and diffuses the received ozone with the fluid, when the fluid is being supplied from the reservoir to a sterilizing mechanism of the apparatus. The corona discharge tube comprises a cylindrical shaped outer electrode; an inner electrode concentrically positioned within the outer electrode; a tube made of a dielectric material, such as but not limited to glass, positioned between the outer electrode and the inner electrode such that an air chamber is formed between the glass tube and the outer electrode; and a micro air pump to circulate air through the air chamber between the glass tube and the outer electrode for generation of ozone.

The ozone generator may further comprise a power source to apply electric potential difference across the outer electrode and the inner electrode, and to supply electric power to the micro air pump.

Another embodiment provides a method of sterilizing a head portion of an apparatus for performing dental hygiene, comprising providing an ozone generator operatively coupled with a base of the apparatus; circulating air through a corona discharge tube of an ozone generator by operating a micro air pump of the ozone generator; applying, simultaneous to circulation of air through the corona tube, an electric potential difference across a set of electrodes of the corona discharge tube to generate ozone; transferring ozone generated by the corona discharge tube to a bubble diffuser operatively coupled to a reservoir of the apparatus to diffuse the received ozone into a fluid stored in the reservoir; and transferring the fluid having diffused ozone around a head portion of the apparatus to sterilize the head portion.

The method may also comprise step of providing a cap like device over the head portion to create space to hold the fluid around the head portion.

The method may also comprise step of generating ultrasonic waves in the fluid held around the head portion of the apparatus by an ultrasonic mechanism operatively coupled to the cap like device.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a side view of a water floss pick showing arrangement of an ozone generator coupled with a base, according to the embodiments herein.

FIG. 2 is an exemplary schematic diagram showing details of an ozone generator including a corona discharge tube, according to the embodiments herein.

FIG. 3 is an exemplary method flow diagram for the method of sterilizing a head portion of an apparatus for performing dental hygiene, according to the embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Flossing devices, such as the ones disclosed by U.S. Pat. Nos. 9,375,299 and 9,770,315, the complete disclosures of which, in their entireties, are herein incorporated by reference, include a sterile-providing component, comprising a mechanism to enhance the cleanliness of the head. The mechanism may include an ultrasonic mechanism that removes bacteria and germs from the head.

The embodiments herein provide an apparatus for performing dental hygiene, which includes mechanism to sterilize a functional portion of the apparatus, such as a head portion, that is prone to growth of bacteria, and a method to sterilize the head portion of the apparatus. The apparatus and method include an ozone generator integrated with a base of the apparatus, to generate small amounts of ozone that is diffused in a fluid using a bubble diffuser. Ozone is known to destroy pathogens, such as bacteria, by breaking through cell membrane of the bacteria. The ozone diffused fluid is transferred to space around the head portion, which is used for performing oral hygiene operations, to sterilize the head portion.

The ozone generator that is integrated with the apparatus for performing dental hygiene is based on a corona discharge tube configured to be small in size so that it may be accommodated on a base of the apparatus. The corona discharge tube comprises a cylindrical shaped outer electrode and an inner electrode with a glass tube positioned between the outer electrode and the inner electrode such that an air chamber is formed between the glass tube and the outer electrode. A micro air pump is integrated with the corona discharge tube to circulate air through the air chamber. When an electric potential difference is applied across the electrodes, a small amount of ozone is generated within the air chamber.

The space around the head portion is created by a cap like device connected to the head portion in watertight manner. An additional sterilizing mechanism, such as an ultrasonic mechanism of the sterilizing mechanism is configured with the cap like device to generate ultrasonic waves through the fluid held around the head portion for sterilizing the head portion.

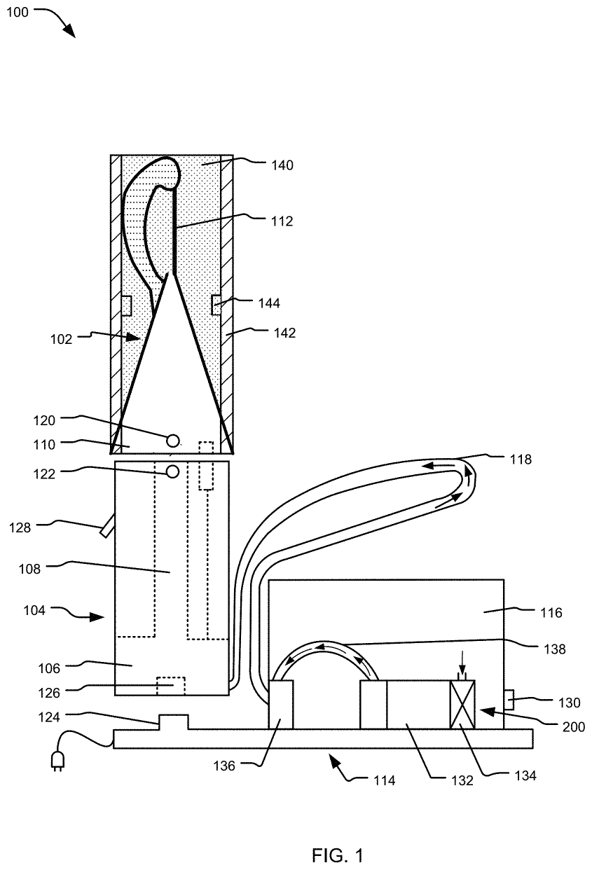

Referring to FIG. 1, where a side view of a water floss pick apparatus 100 for performing oral hygiene is shown incorporating a mechanism to sterilize a functional portion of the apparatus 100 and may include a disposable head portion 102 having a floss attachment 112, and a handle 104 that includes a water pressure motor 106 and an aqueduct 108 (both illustrated in phantom), wherein the aqueduct 108 is aligned with a water intake component 110 of the disposable head portion 102. A base 114 of the apparatus 100 includes a reservoir 116 which holds a fluid used in performing one or more dental hygiene operations, such as water and/or mouthwash and/or any other type of dental cleaning fluid. A water hose 118 connects the reservoir 116 to the handle 104 to enable transfer of water from the reservoir 116 to the water pressure motor 106.

The disposable head 102 and handle 104 each comprise respective complementary secondary alignment and locking features 120 and 122 respectively, that provide alignment of the disposable head 102 with the handle 104 and to provide locking of the disposable head 102 with the handle 104 to prevent inadvertent dislodging of the head portion 102 during use or over time.

When the disposable head portion 102 is inserted into the handle 104, the water intake component 110 is aligned with the aqueduct 108 so as to allow pressurized water (not shown) from the reservoir 116 to flow into the disposable head portion 102 and onto the floss attachment 112 during use.

The base 114 comprises a re-charging contact member 124 and the handle 104 includes a charge contact 126 (shown in phantom) integral with the water pressure motor 106 complementary to the re-charging contact member 124 on the base 114. The handle 104 includes an on/off switch 128, to enable operation of the water pressure motor 106 for the flow of water to the head portion 102 when the head portion 102 is in use; i.e., the handle 104 is lifted off the base 114. The reservoir 114 includes a regulating switch 130, which may be used for other functions of the apparatus 100, such as sterilizing the head portion, which id further described below.

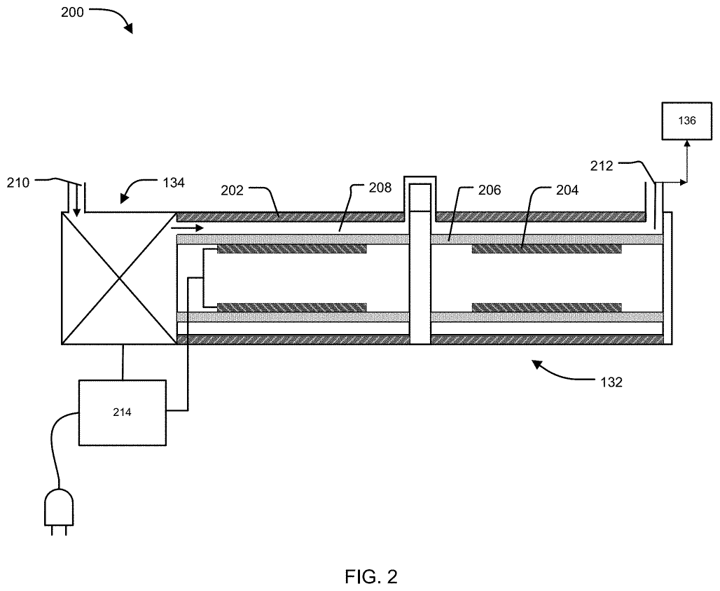

In an embodiment, the base 114 of the apparatus 100 includes an ozone generator 200 (refer to FIG. 2) that generates ozone in small amounts and diffuses the generated ozone in water that is stored in the reservoir 116 for sterilizing the head portion 102 of the apparatus 100. The ozone generator comprises a corona discharge tube 132, a micro air pump 134, and a bubble diffuser 136.

The bubble diffuser 136 may be operatively coupled to the reservoir 116 to selectively diffuse the generated ozone in the water such that ozone is diffused in water only when the water is being withdrawn from the reservoir 116 for sterilizing the head portion 102. This enables to prevent ozone from reaching the head portion 102 when the head portion 102 is being used for performing dental hygiene operation.

In an embodiment, the bubble diffuser 136 may be located at the outlet of the reservoir 116; i.e., at the point where the water hose 118 is connected to the reservoir 116 so that only the water that is moving from the reservoir 116 to the water pressure motor 106 is diffused with ozone, and not all the water stored in the reservoir 116. Thus, operating the ozone generator 200 only when sterilization of the head portion 102 is being performed ensures that ozone does not reach the head portion 102 when the head portion 102 is being used for performing dental hygiene operation.

In an embodiment, sterilization of the head portion 102 using ozone is enabled by filling space around the head portion 102 with water having diffused ozone. The space, such as space 140, may be created using a cap like device 142 configured over the head portion 102. The cap like device 142 may be adapted to fit around the head portion 102 in watertight manner to prevent leakage of water as the space 140 is filled with ozone diffused water.

In an embodiment, the water pressure motor 106 and the aqueduct 108 that is used to move water from the reservoir to the floss attachment 112 may be used for filling the space 140 with the ozone diffused water. Thus, when sterilization of the head portion 102 using ozone is to be performed, both the corona discharge tube 132 along with the micro air pump 134 of the ozone generator 200, along with the water pressure motor 106 may be activated to generate ozone in the corona discharge tube 132, which is diffused in water by the bubble diffuser 136, before being transferred from the reservoir 116 to the head portion 102. The ozone diffused water may overflow from the floss attachment 112 to fill the space 140 around the head portion 102.

In an embodiment, the apparatus 100 may include an additional means for sterilizing the head portion 102, such as an ultrasonic mechanism 144. The ultrasonic mechanism 144 may generate ultrasonic waves through water filled in the space 140 around the head portion 102.

In an embodiment, the regulating switch 130 may be configured to control operation of the sterilizing mechanisms. For example, the regulating switch 130 may have different positions for activating the ultrasonic mechanism 144 based sterilization and the ozone based sterilization, either alone or in combination with the other. For example, the regulating switch 130 may have one position in which only the water pressure motor 106 and the ozone generator 200 are activated to fill ozone diffused water in space 140 around the head portion 102 for sterilizing the head portion 102. In a different position of the regulating switch 130, the water pressure motor 106 and the ozone generator 200 may be activated along with the ultrasonic mechanism 144 to sterilize the head portion 102 by a combination of ozone and the ultrasound waves. In yet another position of the regulating switch 130, the water pressure motor 106 alone may be activated to fill water, which is not diffused with ozone, in the space 140, followed by activation of the ultrasonic mechanism 144 to sterilize the head portion 102 by the ultrasound waves alone.

In an implementation, in either application, the water pressure motor 106 may be operated for a predefined time to transfer just enough water from the reservoir 116 to the space 140 so that the space 140 is adequately filled. Alternatively, there may be provision for overflow of the excess water from the space 140.

FIG. 2, with reference to FIG. 1, shows details of the ozone generator 200 based on the corona discharge tube, according to the embodiments herein. The corona discharge tube 132 may include a cylindrical shaped outer electrode 202; an inner electrode 204 concentrically positioned within the outer electrode; and a tube 206 made of a dielectric material, such as but not limited to glass. The glass tube 206 may be positioned between the outer electrode 202 and the inner electrode 204 such that an air chamber 208 is formed between the glass tube 206 and the outer electrode 202. A potential difference may be applied across the outer electrode 202 and the inner electrode 204 to generate ozone from oxygen in air that lies in the air chamber 208. In an embodiment, the outer electrode 202 and the inner electrode 204 may be made of a suitable conductive material such as, but not limited to stainless steel.

In an embodiment, the micro air pump 134 to circulate air through the air chamber 208 and onward transfer to the bubble diffuser 136, may be coupled to the corona discharge tube 132 at one side to make a compact unit that can be accommodated on the base 114. The micro air pump 134 may intake air through air inlet 210, and the air mixed with the generated ozone may flow out of the air chamber 208 through an air outlet 212, which is connected to the bubble diffuser 136 through an air pipe 138, as shown in FIG. 1.

The ozone generator may further comprise a power source 214 to apply electric potential difference across the outer electrode 202 and the inner electrode 204, and to supply electric power to the micro air pump 134. In an embodiment, the power source 214 may be a common source that supplies electric power for the water pressure motor 106.

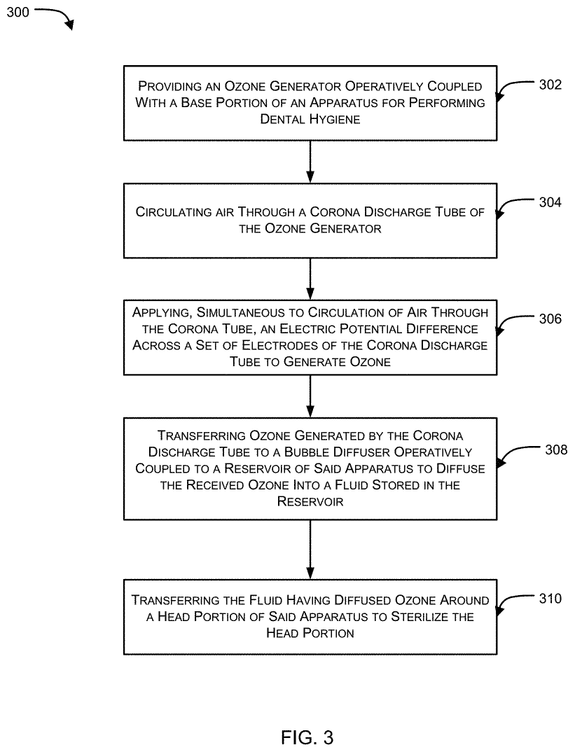

FIG. 3, with reference to FIGS. 1 and 2, is an exemplary method flow diagram for the method 300 of sterilizing a head portion, such as head portion 102 of an apparatus 100 for performing dental hygiene, such as apparatus 100 shown in FIG. 1. The method 300 comprises, at step 302, providing an ozone generator, such as ozone generator 200 shown in FIG. 2, operatively coupled with a base, such as base 114 shown in FIG. 1 of the apparatus 100.

At step 304 of the method 300, air may be circulated through a corona discharge tube, such as corona discharge tube 132 shown in FIGS. 1 and 2, of the ozone generator 200, by operating a micro air pump, such as micro pump 134 shown in FIGS. 1 and 2, of the ozone generator 200. The corona discharge tube 132 and the micro pump 134 may be operatively coupled with a base, such as base 114 shown in FIG. 1, of the apparatus 100.

At step 306 of the method 300, to generate ozone, an electric potential difference may be applied across a set of electrodes such as the outer electrode 202 and the inner electrode 204 shown in FIG. 2, of the corona discharge tube 132, which is performed simultaneous to circulation of air through the corona discharge tube 132.

At step 308 of the method 300, ozone generated by the corona discharge tube 132 is transferred to a bubble diffuser, such as the bubble diffuser 136 shown in FIGS. 1 and 2, that is operatively coupled to a reservoir of the apparatus 100, such as reservoir 116 shown in FIG. 1, to diffuse the received ozone into a fluid stored in the reservoir 116.

At step 310 of the method 300, the fluid having diffused ozone may be transferred to a space, such as space 140 shown in FIG. 1, that surrounds the head portion 102 of the apparatus 100 to sterilize the head portion 102. This can be performed by a water pressure motor, such as water pressure motor 106, shown in FIG. 1.

In an embodiment, the method 300 may also comprise a step of providing a cap like device, such as cap like device 142 shown in FIG. 1, over the head portion 102 to create space 140 that holds the fluid with diffused ozone around the head portion 102.

In an embodiment, the method 300 may also comprise a step of generating ultrasonic waves in the fluid held in space 140 around the head portion 102 of the apparatus 100 by an ultrasonic mechanism, such as ultrasonic mechanism 144 shown in FIG. 1, that is operatively coupled to the cap like device 142.

Thus, the embodiments herein provide an apparatus 100 for performing dental hygiene, which includes a mechanism to sterilize a functional portion of the apparatus, such as a head portion 102, that is prone to growth of bacteria, and a method 300 to sterilize the head portion 102 of the apparatus 100. The apparatus 100 includes an ozone generator 200 integrated with a base 114 of the apparatus 100 to generate small amounts of ozone that is diffused in a fluid using a bubble diffuser 136. The ozone diffused fluid is transferred to a space 140 around the head portion 102 to sterilize the head portion 102.

The ozone generator 200 includes a corona discharge tube 132 configured to be small in size so that it may be accommodated on the base 114 of the apparatus 100. The corona discharge tube 132 comprises a cylindrical shaped outer electrode 202 and an inner electrode 204 with a glass tube 206 positioned between the outer electrode 202 and the inner electrode 204 such that an air chamber 208 is formed between the glass tube 206 and the outer electrode 202. A micro air pump 134 is integrated with the corona discharge tube 132 to circulate air through the air chamber 208. When an electric potential difference is applied across the electrodes 202/204, a small amount of ozone is generated within the air chamber 208.

The space 140 around the head portion is created by a cap like device 142 connected to the head portion in watertight manner. An additional sterilizing mechanism, such as an ultrasonic mechanism 144 may be configured with the cap like device 142 to generate ultrasonic waves through the fluid held around the head portion 102 for sterilizing the head portion 102.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein may be practiced with modification within the spirit and scope of the appended claims.

What is claimed is:

1. An apparatus for performing dental hygiene comprising a multi-piece body component comprising:
a head portion configured for performing one or more dental hygiene operations;
a sterilizing mechanism operatively connected to the head portion that reduces bacteria from the head portion, wherein the sterilizing mechanism comprises an ultrasonic mechanism for reducing bacteria; and
a base comprising;
a reservoir to store a fluid used during the one or more dental hygiene operations and by the sterilizing mechanism to reduce bacteria from the head portion;
a corona discharge tube to generate ozone; and
a bubble diffuser operatively coupled to the reservoir, wherein the bubble diffuser receives ozone from the corona discharge tube and diffuses the received ozone with the fluid when the fluid is being supplied from the reservoir to the sterilizing mechanism.

2. The apparatus of claim 1, wherein the corona discharge tube comprises:
a cylindrical shape outer electrode;
an inner electrode concentrically positioned within the outer electrode;
a tube made of a dielectric material positioned between the outer electrode and the inner electrode such that an air chamber is formed between the glass tube and the outer electrode; and
a micro air pump to circulate air through the air chamber between the glass tube and the outer electrode.

3. The apparatus of claim 2, wherein the dielectric material comprises glass.

4. The apparatus of claim 2, comprising a power source to apply an electric potential difference across the outer electrode and the inner electrode, and to supply electric power to the micro air pump.

5. The apparatus of claim 1, comprising a cap like device operatively connected to the head portion, wherein the sterilizing mechanism is configured with the cap like device.

6. The apparatus of claim 5, wherein the cap like device fits the head portion in a watertight manner to enable circulation of the fluid from the reservoir around the head portion.

7. The apparatus of claim 1, wherein the ultrasonic mechanism of the sterilizing mechanism generates ultrasonic waves through the fluid circulating around the head portion.

8. An ozone generator for an apparatus for performing dental hygiene, comprising:
a corona discharge tube operatively coupled to a base of the apparatus, comprising:
a cylindrical shaped outer electrode;
an inner electrode concentrically positioned within the outer electrode;
a tube made of a dielectric material positioned between the outer electrode and the inner electrode such that an air chamber is formed between the glass tube and the outer electrode; and
a micro air pump to circulate air through the air chamber between the glass tube and the outer electrode for generation of ozone;
a bubble diffuser operatively coupled to a reservoir of the apparatus that holds a fluid used in the apparatus during dental hygiene operation, wherein the bubble diffuser is operatively coupled to an outlet of the corona discharge tube to receive the generated ozone from the corona discharge tube and diffuses the received ozone with the fluid when the fluid is being supplied from the reservoir to a sterilizing mechanism of the apparatus.

9. The ozone generator of claim 8, wherein the dielectric material comprises glass.

10. The ozone generator of claim 8, comprising a power source to apply electric potential difference across the outer electrode and the inner electrode, and to supply electric power to the micro air pump.

11. An apparatus for performing dental hygiene comprising a multi-piece body component comprising:
a head portion used for performing one or more dental hygiene operations;
a cap like device adapted to fit the head portion in a watertight manner to create space around the head portion for holding a fluid;
a base comprising:
a reservoir to store the fluid;
a corona discharge tube to generate ozone; and
a bubble diffuser operatively coupled to the reservoir, wherein the bubble diffuser receives ozone from the corona discharge tube and diffuses the received ozone with the fluid.

12. The apparatus of claim 11, wherein the corona discharge tube comprises:
a cylindrical shape outer electrode;
an inner electrode concentrically positioned within the outer electrode;
a tube made of a dielectric material positioned between the outer electrode and the inner electrode such that an air chamber is formed between the glass tube and the outer electrode; and
a micro air pump to circulate air through the air chamber between the glass tube and the outer electrode.

13. The apparatus of claim 12, wherein the dielectric material comprises glass.

14. The apparatus of claim 12, comprising a power source to apply electric potential difference across the outer electrode and the inner electrode, and to supply electric power to the micro air pump.

15. The apparatus of claim 11, comprising a sterilizing mechanism operatively connected to the cap like device to reduces bacteria from the head portion, wherein the sterilizing mechanism comprises an ultrasonic mechanism for reducing bacteria.

16. The apparatus of claim 11, wherein the ultrasonic mechanism of the sterilizing mechanism generates ultrasonic waves through the fluid held around the head portion.

17. The apparatus of claim 11, wherein the fluid stored in the reservoir is also used by the apparatus for performing dental hygiene operations.
